(19) United States
(12) Patent Application Publication
(10) Pub. No.: US 2021/0172005 A1
(43) Pub. Date: Jun. 10, 2021

(54) INFECTION-RELATED PRETERM BIRTH DIAGNOSTIC METHOD

(71) Applicant: The University of Western Australia, Nedlands (AU)

(72) Inventors: Jeffrey Keelan, Stirling, Western Australia (AU); Matthew Scott Payne, Landsdale, Western Australia (AU)

(21) Appl. No.: 16/771,733

(22) PCT Filed: Nov. 22, 2018
(86) PCT No.: PCT/AU2018/051249

§ 371 Date: Jun. 11, 2020

(30) Foreign Application Priority Data
Nov. 24, 2017 (AU) 2017904748
Sep. 20, 2018 (AU) 2018903531

(51) Int. Cl. C12Q 1/689; C12Q 1/04

(52) U.S. Cl. CPC C12Q 1/689; C12Q 1/04; G01N 2333/30; C12Q 2600/118; G01N 2333/40; G01N 2800/368; G01N 2333/335

(57) ABSTRACT

A method to determine if a pregnant woman is at risk of infection-associated spontaneous pre-term birth (sPTB), the method comprising the steps of: b) testing a sample of vaginal fluid for the presence of the following bacteria: iv) Ureaplasma parvum genotype SV3 and/or Ureaplasma parvum genotype SV6; v) Gardnerella vaginalis ; and vi) Lactobacillus iners wherein the presence of the bacteria indicates that the subject is at risk of sPTB.

### TECHNICAL FIELD

The present invention relates to methods and kits for the diagnosis of pregnancies at risk of preterm birth (PTB) due to ascending intrauterine infection.

### BACKGROUND ART

Despite decades of research, and major advances in understanding of aetiology, PTB remains a major obstetric healthcare problem of national and global significance. PTB is the single major cause of death and disability in children up to five years of age in the developed world, and the leading single cause of perinatal mortality and morbidity; approximately 15 million babies are born preterm each year world-wide. While many children born too early go on to lead a normal and healthy life, a significant proportion do not survive or experience life-long disability; the impact on individuals, families and society are considerable, as are the healthcare costs associated with perinatal care and life-long disability.

Around 20% of spontaneous PTB (sPTB) cases are due to intrauterine infection, which in the majority arises from ascension of bacteria from the vaginal mucosa into the pregnant uterus, eliciting chorioamnionitis, funisitis and preterm labour and delivery. While multiple bacteria have been found to cause PTB, identifying women early in pregnancy at risk in order to receive appropriate preventative treatment has been problematic. Traditional microbiological methods are imprecise, do not have a high predictive potential, and require skilled interpretation (which is a slow process and the number of suitably skilled interpreters is not large, thus they are not widely performed).

Trials to prevent sPTB using prophylactic antibiotic administration have had mixed success. In order to achieve maximal benefits in terms of PTB reduction, antimicrobial treatment should be applied selectively to women at risk based upon vaginal microbial status, while avoiding unnecessary treatment of women at low microbial risk. Typically, women have been recruited into trials based on the diagnosis of bacterial vaginosis (BV) or other related vaginal microbial risk factors. This is because the presence of BV has been the only means of identifying women at risk of sPTB based on vaginal microbiology.

A large number of microorganisms have been implicated in the aetiology of sPTB. Some of the bacteria that regularly cause infection-driven sPTB are common bacteria frequently found in the reproductive tract of pregnant women, while others are only found in women with abnormal vaginal microbiota (e.g. BV) and/or are associated with reproductive tract infections. In up to half of infection-associated sPTBs, multiple bacteria are present in the amniotic cavity. The microorganism most commonly associated with sPTB is Ureaplasma, a genus of intracellular bacteria that are present in the reproductive tract of approximately half of pregnant women, independent of other markers of vaginal dysbiosis. Studies have shown that the presence of Ureaplasma (usually not defined at the species level) is a weak risk factor for sPTB.

The identification of women at risk of an infection-related preterm delivery is far from simple, and to date has relied on imprecise diagnosis of conditions such as BV. BV is characterized by a disturbance of normal vaginal microbiota, a loss of H₂O₂-producing Lactobacillus spp., an increase in vaginal pH, and an increase in Gram-variable cocco-bacilli, anaerobic organisms and genital mycoplasmas. Importantly, it is known that the vaginal microbiota associated with BV varies according to race. BV has been shown to be predictive of increased risk of sPTB in populations with African ethnicities, but is a relatively weak risk predictor in Caucasian populations (OR <2) with a low prevalence rate (<10%). Aerobic vaginitis (AV) is also a risk factor for sPTB, with a similar risk profile as BV, albeit with different microbial characteristics.

Identification of women at risk and who will respond to treatment is a critical factor in the design of successful interventions to prevent infection-related sPTB. There is, therefore, a need for alternative sPTB diagnostic methods, or at least the provision of new diagnostic methods to compliment the previously known methods.

To date, the identification of women at risk of sPTB based on their Ureaplasma status has not been possible, despite the fact that it is the microorganism most commonly found in infected preterm deliveries and is readily treatable. This is a major weakness of current sPTB prediction methods.

The present invention seeks to provide an improved or alternative method for the diagnosis of pregnancies at risk of infection-driven sPTB based on a microbiological profile including assessment of Ureaplasma colonisation status, so that appropriate preventative treatment can be applied and targeted to at-risk women.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a method to determine if a pregnant woman is at risk of infection-associated spontaneous preterm birth (sPTB), the method comprising the steps of:
a) testing a sample of vaginal fluid for the presence of the following bacteria:
   i) Ureaplasma parvum genotype SV3 and/or Ureaplasma parvum genotype SV6;
   ii) Gardnerella vaginalis ; and
   iii) Lactobacillus iners
      wherein the presence of the bacteria indicates that the subject is at risk of a sPTB.

Optionally, the testing method further tests for the presence of Fusobacterium nucleatum, wherein the presence of either:

Fusobacterium nucleatum in the absence of Ureaplasma parvum genotype SV3 and/or Ureaplasma parvum genotype SV6; or

Ureaplasma parvum genotype SV3 and/or Ureaplasma parvum genotype SV6, Gardnerella vaginalis, and Lactobacillus iners

indicates that the subject is at risk of a sPTB.

Preferably, the testing method is quantitative PCR (qPCR).

The testing may optionally be preceded by testing for the presence of high levels of a Lactobacillus species other than L. iners, preferably Lactobacillus gasseri, Lactobacillus crispatus and/or Lactobacillus jensenii. If high levels of these Lactobacillus species are detected, then the risk of infection-associated spontaneous preterm birth (sPTB) is low and step (a) need not be carried out.

The invention further provides a method to determine if a pregnant woman would benefit from treatment to prevent infection-associated sPTB, the method comprising the steps of:
a) testing a sample of vaginal fluid for the presence of the following bacteria:
   i) Ureaplasma parvum genotype SV3 and/or Ureaplasma parvum genotype SV6;
   ii) Gardnerella vaginalis; and
   iii) Lactobacillus iners
      wherein the presence of the bacteria indicates that the subject is at risk of a sPTB and therefore would benefit from treatment to prevent a sPTB.

The invention further provides a method to treat a pregnant woman at risk of infection-associated sPTB, comprising the steps of:
a) testing a sample of vaginal fluid for the presence of the following bacteria:
   i) Ureaplasma parvum genotype SV3 and/or Ureaplasma parvum genotype SV6;
   ii) Gardnerella vaginalis ; and
   iii) Lactobacillus iners
b) if the bacteria are present, giving the pregnant woman antibiotic therapy to eliminate the bacteria.

The invention further provides a method of reducing the risk of a pregnant woman having an infection-associated sPTB, comprising the steps of:
a) testing a sample of vaginal fluid for the presence of the following bacteria:
   i) Ureaplasma parvum genotype SV3 and/or Ureaplasma parvum genotype SV6;
   ii) Gardnerella vaginalis; and
   iii) Lactobacillus iners
b) if the bacteria are present, giving the pregnant woman antibiotic therapy to eliminate the bacteria and therefore reduce the risk of sPTB.

The antibiotic therapy may optionally be followed by or administered concurrently with a probiotic therapy to reduce the chance of re-infection.

The present invention provides a kit to determine if a pregnant woman is at risk of an infection-associated sPTB, the kit comprising:
a) a means for testing a sample of vaginal fluid for the presence of the following bacteria:
   i) Ureaplasma parvum genotype SV3 and/or Ureaplasma parvum genotype SV6;
   ii) Gardnerella vaginalis; and
   iii) Lactobacillus iners
b) Instructions for use.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a Venn diagram showing the differences between sPTB detection rates based on a molecular diagnosis of BV versus a diagnosis using the GLU (Gardnerella vaginalis; Lactobacillus iners; Ureaplasma parvum genotype SV3 or SV6; Fusobacterium nucleatum) PCR assay of the present invention. BV is defined as positive for G. vaginalis and two or more additional BV-associated bacteria.

FIG. 2 is a graph of PTB prediction showing the differences between sPTB detection rates, sub-divided by gestational age at delivery, based on a molecular diagnosis of BV versus a diagnosis using the GLU test of the present invention.

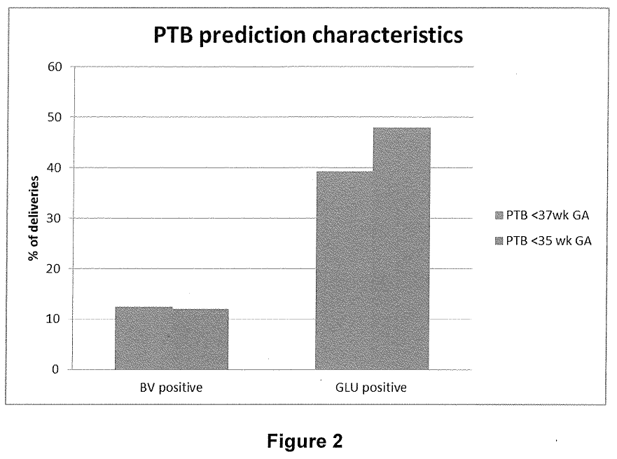

FIG. 3 is a graph of the stability of prevalence of Ureaplasma, Candida and Mycoplasma spp. in vaginal swabs from 134 women with three completed samples taken over the course of the pregnancy. Solid black, sample time point 1; dark grey, sample time point 2; light grey, sample time point 3.

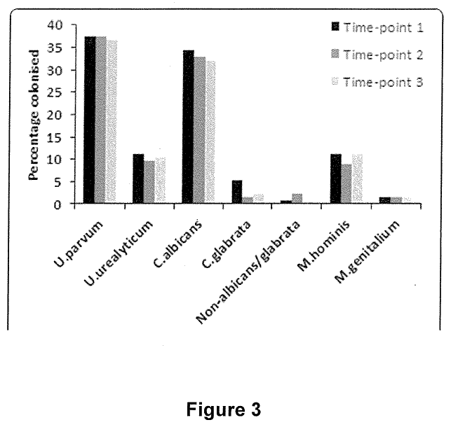

FIG. 4 is a flow chart of the timeline for Example 3: Clinical trial of a “screen and treat” program.

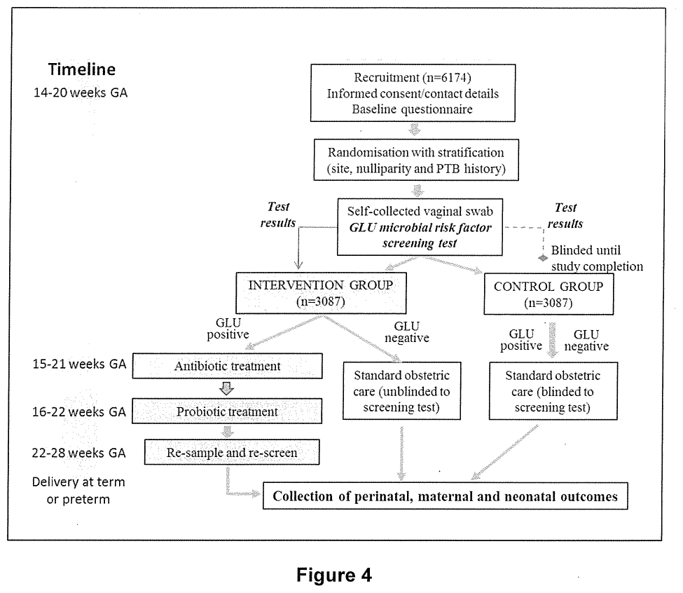

### DESCRIPTION OF INVENTION

### Detailed Description of the Invention

Method of Detection

There is a global need for reliable, rapid, inexpensive, simple and effective methods to diagnose women at risk of infection-associated spontaneous pre-term birth (sPTB) in early-/mid-pregnancy and establish which populations of pregnant women would most benefit from antibiotic therapy to reduce this risk. The present invention can be used to identify a significant proportion of women at risk of infection-associated sPTB in the first half of pregnancy, so that targeted antimicrobial and probiotic therapy can be applied to eliminate the bacteria and reduce the rates of sPTB.

Therefore, the present invention provides a method to determine if a pregnant woman is at risk of infection-associated sPTB, the method comprising the steps of:
a) testing a sample of vaginal fluid for the presence of the following bacteria:
   i) Ureaplasma parvum genotype SV3 and/or Ureaplasma parvum genotype SV6;
   ii) Gardnerella vaginalis; and
   iii) Lactobacillus iners
      wherein the presence of the bacteria indicates that the subject is at risk of a sPTB.

The invention further provides a method to determine if a pregnant woman would benefit from treatment to prevent infection-associated sPTB, the method comprising the steps of:
a) testing a sample of vaginal fluid for the presence of the following bacteria:
   i) Ureaplasma parvum genotype SV3 and/or Ureaplasma parvum genotype SV6;
   ii) Gardnerella vaginalis; and
   iii) Lactobacillus iners
      wherein the presence of the bacteria indicates that the subject is at risk of a sPTB and therefore would benefit from treatment to prevent PTB.

The antibiotic therapy may optionally be followed by or administered concurrently with a probiotic therapy to reduce the chance of re-infection.

Preterm birth is defined by the World Health Organization (WHO) as babies born alive before 37 weeks' of pregnancy are completed. There are sub-categories of PTB, based on gestational age, which are:

extremely preterm (<28 weeks)

very preterm (28 to <34 weeks)

moderate to late preterm (35 weeks+).

The present invention has found that the incidence of sPTB is higher in pregnant women who test positive for the bacterial profile listed above. Without being held to any theory, we believe that pregnant women who test positive with the aforementioned bacterial signature are more likely to benefit from antimicrobial therapy to reduce the risk of a sPTB.

The presence of three or more of the list bacteria may be four bacteria from the list.

Ureaplasma is associated with most cases of sPTB, but most women with Ureaplasma aren't at risk of sPTB. Prior to the present invention, there was no way of identifying those pregnant women who are or are not at risk. Previous tests involving BV diagnosis have ignored Ureaplasma status, as it is not a BV-related organism. Furthermore, until now, detection has generally been limited to identification to the genus level of human Ureaplasma species, and has not differentiated the two known species and associated serovars. Preferably the infection-associated sPTB is associated with ascending intrauterine infection; transfer across the placenta in the mother's blood; infection introduced by invasive procedures such as amniocentesis, and colonisation of the non-pregnant uterus by bacteria. Most preferably, the infection-associated sPTB is associated with ascending intrauterine infection.

Optionally, the present invention provides a method to determine if a pregnant woman is at risk of infection-associated sPTB, the method comprising the steps of:
a) testing a sample of vaginal fluid for the presence of the following bacteria:
   i) Fusobacterium nucleatum;
   ii) Ureaplasma parvum genotype SV3 and/or Ureaplasma parvum genotype SV6;
   iii) Gardnerella vaginalis; and
   iv) Lactobacillus iners
      wherein the presence of either:

Fusobacterium nucleatum in the absence of Ureaplasma parvum genotype SV3 and/or Ureaplasma parvum genotype SV6; or

Ureaplasma parvum genotype SV3 and/or Ureaplasma parvum genotype SV6, Gardnerella vaginalis, and Lactobacillus iners

indicates that the subject is at risk of a sPTB.

Optionally, the present invention provides a method to determine if a pregnant woman would benefit from treatment to prevent infection-associated sPTB, the method comprising the steps of:
a) testing a sample of vaginal fluid for the presence of the following bacteria:
   i) Fusobacterium nucleatum;
   ii) Ureaplasma parvum genotype SV3 and/or Ureaplasma parvum genotype SV6;
   iii) Gardnerella vaginalis; and
   iv) Lactobacillus iners
      wherein the presence of either:
   Fusobacterium nucleatum in the absence of Ureaplasma parvum genotype SV3 and/or Ureaplasma parvum genotype SV6; or
   Ureaplasma parvum genotype SV3 and/or Ureaplasma parvum genotype SV6, Gardnerella vaginalis, and Lactobacillus iners
      indicates that the subject is at risk of a sPTB.

Optionally, the Gardnerella vaginalis tested for is clade 4.

Without being held to any theory, it is believed that the presence of Fusobacterium nucleatum may itself impart an increased risk of infection-associated sPTB. As the risk of sPTB is already high in women with Ureaplasma parvum genotype SV3 and/or SV6, the additional predictive power added by the presence of F. nucleatum is only significant in women who are negative for U. parvum SV3/SV6.

The risks therefore segregate as follows:

| | Infection- |
|---|---|
| | associated |
| Bacterial Presence | sPTB Risk |
| Ureaplasma parvum genotype SV3 and/or SV6: +ve | High |
| Gardnerella vaginalis: +ve | |
| Lactobacillus iners: +ve | |
| Ureaplasma parvum genotype SV3 and/or SV6: +ve | Low |
| Gardnerella vaginalis: −ve | |
| Lactobacillus iners: +ve | |
| Ureaplasma parvum genotype SV3 and/or SV6: +ve | Low |
| Gardnerella vaginalis: +ve | |
| Lactobacillus iners: −ve | |
| Ureaplasma parvum genotype SV3 and/or SV6: +ve | Low |
| Gardnerella vaginalis: −ve | |
| Lactobacillus iners: −ve | |
| Ureaplasma parvum genotype SV3 and/or SV6: −ve | Low |
| Gardnerella vaginalis: +ve | |
| Lactobacillus iners: +ve | |
| Ureaplasma parvum genotype SV3 and/or SV6: −ve | Low |
| Gardnerella vaginalis: −ve | |
| Lactobacillus iners: +ve | |
| Ureaplasma parvum genotype SV3 and/or SV6: −ve | Low |
| Gardnerella vaginalis: +ve | |
| Lactobacillus iners: −ve | |
| Ureaplasma parvum genotype SV3 and/or SV6: −ve | Low |
| Gardnerella vaginalis: −ve | |
| Lactobacillus iners: −ve | |
| Fusobacterium nucleatum: +ve | High |
| Ureaplasma parvum genotype SV3 and/or SV6: +ve | |
| Gardnerella vaginalis: +ve | |
| Lactobacillus iners: +ve | |
| Fusobacterium nucleatum: +ve | Low |
| Ureaplasma parvum genotype SV3 and/or SV6: +ve | |
| Gardnerella vaginalis: −ve | |
| Lactobacillus iners: +ve | |
| Fusobacterium nucleatum: +ve | Low |
| Ureaplasma parvum genotype SV3 and/or SV6: +ve | |
| Gardnerella vaginalis: +ve | |
| Lactobacillus iners: −ve | |
| Fusobacterium nucleatum: +ve | Low |
| Ureaplasma parvum genotype SV3 and/or SV6: +ve | |
| Gardnerella vaginalis: −ve | |
| Lactobacillus iners: −ve | |
| Fusobacterium nucleatum: +ve | High |
| Ureaplasma parvum genotype SV3 and/or SV6: −ve | |
| Gardnerella vaginalis: +ve | |
| Lactobacillus iners: +ve | |
| Fusobacterium nucleatum: +ve | High |
| Ureaplasma parvum genotype SV3 and/or SV6: −ve | |
| Gardnerella vaginalis: −ve | |
| Lactobacillus iners: +ve | |
| Fusobacterium nucleatum: +ve | High |
| Ureaplasma parvum genotype SV3 and/or SV6: −ve | |
| Gardnerella vaginalis: +ve | |
| Lactobacillus iners: −ve | |
| Fusobacterium nucleatum: +ve | High |
| Ureaplasma parvum genotype SV3 and/or SV6: −ve | |
| Gardnerella vaginalis: −ve | |
| Lactobacillus iners: −ve | |
| Fusobacterium nucleatum: −ve | High |
| Ureaplasma parvum genotype SV3 and/or SV6: +ve | |
| Gardnerella vaginalis: +ve | |
| Lactobacillus iners: +ve | |
| Fusobacterium nucleatum: −ve | Low |
| Ureaplasma parvum genotype SV3 and/or SV6: +ve | |
| Gardnerella vaginalis: −ve | |
| Lactobacillus iners: +ve | |
| Fusobacterium nucleatum: −ve | Low |
| Ureaplasma parvum genotype SV3 and/or SV6: +ve | |
| Gardnerella vaginalis: +ve | |
| Lactobacillus iners: −ve | |
| Fusobacterium nucleatum: −ve | Low |
| Ureaplasma parvum genotype SV3 and/or SV6: +ve | |
| Gardnerella vaginalis: −ve | |
| Lactobacillus iners: −ve | |
| Fusobacterium nucleatum: −ve | Low |
| Ureaplasma parvum genotype SV3 and/or SV6: −ve | |
| Gardnerella vaginalis: +ve | |
| Lactobacillus iners: +ve | |
| Fusobacterium nucleatum: −ve | Low |
| Ureaplasma parvum genotype SV3 and/or SV6: −ve | |
| Gardnerella vaginalis: −ve | |
| Lactobacillus iners: +ve | |
| Fusobacterium nucleatum: +ve | High |
| Ureaplasma parvum genotype SV3 and/or SV6: −ve | |
| Gardnerella vaginalis: +ve | |
| Lactobacillus iners: −ve | |
| Fusobacterium nucleatum: −ve | Low |
| Ureaplasma parvum genotype SV3 and/or SV6: −ve | |
| Gardnerella vaginalis: −ve | |
| Lactobacillus iners: −ve | |

Therefore, a pregnant woman who tests positive for the following combinations of bacterial species are at high risk for an infection-associated sPTB:
Ureaplasma parvum genotype SV3 and/or SV6: +ve; Gardnerella vaginalis: +ve; Lactobacillus iners: +ve
Fusobacterium nucleatum : +ve; Ureaplasma parvum genotype SV3 and/or SV6: +ve; Gardnerella vaginalis: +ve; Lactobacillus iners: +ve
Fusobacterium nucleatum: +ve; Ureaplasma parvum genotype SV3 and/or SV6: −ve; Gardnerella vaginalis: +ve; Lactobacillus iners: +ve
Fusobacterium nucleatum: +ve; Ureaplasma parvum genotype SV3 and/or SV6: −ve; Gardnerella vaginalis: −ve; Lactobacillus iners: +ve
Fusobacterium nucleatum: +ve; Ureaplasma parvum genotype SV3 and/or SV6: −ve; Gardnerella vaginalis: +ve; Lactobacillus iners: −ve
Fusobacterium nucleatum: +ve; Ureaplasma parvum genotype SV3 and/or SV6: −ve; Gardnerella vaginalis: −ve; Lactobacillus iners: −ve
Fusobacterium nucleatum: −ve; Ureaplasma parvum genotype SV3 and/or SV6: +ve; Gardnerella vaginalis: +ve; Lactobacillus iners: +ve
Fusobacterium nucleatum: +ve; Ureaplasma parvum genotype SV3 and/or SV6: −ve; Gardnerella vaginalis: +ve; Lactobacillus iners: −ve
   and other combinations are low risk.

Optionally, the Gardnerella vaginalis tested for is clade 4.

Optionally, the testing may be preceded by testing for the presence of high levels of Lactobacillus species other than Lactobacillus iners in the vaginal fluid. If high levels of Lactobacillus species other than Lactobacillus iners are detected, then the risk of infection-associated spontaneous preterm birth (sPTB) is low and step (a) need not be carried out. The Lactobacillus species other than Lactobacillus iners are preferably chosen from the list comprising Lactobacillus gasseri, L. crispatus and L. jensenii. By “high levels”, it is meant that there are more than about 10,000 copies, 15,000 copies or preferably more than about 20,000 copies of the 16S rRNA gene of the Lactobacillus species. Alternatively, the presence of high levels of a Lactobacillus species other than Lactobacillus iners may be tested by determining the number of copies of the elongation factor Tu (tuf) gene. Other genes that can be used to quantify the presence of high levels of a Lactobacillus species other than Lactobacillus iners may also be used.

Without being held to any theory, it is believed that high levels of Lactobacillus species other than Lactobacillus iners affords a pregnant woman some protection against the risk of infection-associated sPTB associated with Fusobacterium nucleatum; Ureaplasma parvum genotype SV3 and/or Ureaplasma parvum genotype SV6; Gardnerella vaginalis; and Lactobacillus iners. Therefore, if high levels of Lactobacillus species other than Lactobacillus iners are detected, the risk of infection-associated sPTB associated with Fusobacterium nucleatum; Ureaplasma parvum genotype SV3 and/or Ureaplasma parvum genotype SV6; Gardnerella vaginalis; and Lactobacillus iners is low and step (a) of the above method need not be carried out.

Preferably the testing method is quantitative PCR (qPCR), also known as real-time PCR. Alternatively, the testing may be via endpoint PCR and subsequent DNA sequencing, digital PCR, fluorescence in situ hybridisation, bacterial culture, or immunological testing. In the case of culture, a second round of testing, such as qPCR testing, may be carried out on samples determined via a first method to contain Ureaplasma (in order to identify the species and specific U. parvum genotype) and/or Gardnerella vaginalis (in order to identify the clade).

Preferably the testing is carried out at between 10 and 24 weeks' gestation, between 16 and 24 weeks' gestation, more preferably between 18 and 22 weeks' gestation, most preferably between 18 and 20 weeks' gestation, or before 22 weeks' gestation.

Preferably the fluid collected for sampling is vaginal fluid, also known as cervico-vaginal fluid. Alternatively, the fluid collected may be cervical fluid, cervical mucous and/or material from the cervical mucous plug.

Preferably the sample is self-collected vaginal fluid. For example, the sample may be self-collected using a vaginal swab. Alternatively, the vaginal fluid may be collected in a surgical, hospital or clinical setting. For example, the vaginal fluid, cervical mucous and/or material from the cervical mucous plug could be collected by a health care provider, using a vaginal swab with/without a speculum. The sample may further be a sample of douche fluid collected following vaginal douching.

The vaginal swabs may be dry swabs. Preferably the dry swabs are immediately placed into liquid media.

For the purposes of comparisons with the present invention, BV is defined in the present invention as qPCR detection of G. vaginalis DNA in the cervicovaginal sample plus one or more additional BV-associated bacteria (either F. nucleatum, L. amnionii, S. sanguinegens, M. hominis, Peptostreptococcus spp.).

The test may prove positive for the following combinations of bacteria:
Ureaplasma parvum genotype SV3, Ureaplasma parvum genotype SV6, Gardnerella vaginalis and Lactobacillus iners;
Ureaplasma parvum genotype SV6, Gardnerella vaginalis and Lactobacillus iners;
Ureaplasma parvum genotype SV3, Gardnerella vaginalis and Lactobacillus iners;
Fusobacterium nucleatum; Ureaplasma parvum genotype SV3 and SV6; Gardnerella vaginalis; Lactobacillus iners;
Fusobacterium nucleatum;
Fusobacterium nucleatum; Gardnerella vaginalis; Lactobacillus iners;
Fusobacterium nucleatum; Lactobacillus iners;
Fusobacterium nucleatum; Gardnerella vaginalis;
Fusobacterium nucleatum; Ureaplasma parvum genotype SV3; Gardnerella vaginalis; Lactobacillus iners;
Fusobacterium nucleatum; Ureaplasma parvum genotype SV6; Gardnerella vaginalis; Lactobacillus iners.

### Method of Treatment

The invention further provides a method to treat a pregnant woman at risk of infection-associated sPTB, the method comprising the steps of:
a) testing a sample of vaginal fluid for the presence of the following bacteria:
   i) Ureaplasma parvum genotype SV3 and/or Ureaplasma parvum genotype SV6;
   ii) Gardnerella vaginalis; and
   iii) Lactobacillus iners
b) if the bacteria are present, giving the pregnant woman antibiotic therapy to eliminate the bacteria.

The invention further provides a method of reducing the risk of a pregnant woman having a sPTB, the method comprising the steps of:
a) testing a sample of vaginal fluid for the presence of the following bacteria:
   i) Ureaplasma parvum genotype SV3 and/or Ureaplasma parvum genotype SV6;
   ii) Gardnerella vaginalis; and
   iii) Lactobacillus iners
b) if the bacteria are present, giving the pregnant woman antibiotic therapy to eliminate the bacteria and therefore reduce the risk of sPTB.

The invention further provides a method to treat a pregnant woman at risk of an infection-associated sPTB, the method comprising the steps of:
a) testing a sample of vaginal fluid for the presence of the following bacteria:
   i) Fusobacterium nucleatum;
   ii) Ureaplasma parvum genotype SV3 and/or Ureaplasma parvum genotype SV6;
   iii) Gardnerella vaginalis; and
   iv) Lactobacillus iners
b) if either:
   Fusobacterium nucleatum; or
   Ureaplasma parvum genotype SV3 and/or Ureaplasma parvum genotype SV6, Gardnerella vaginalis, and Lactobacillus iners
      are present, giving the pregnant woman antibiotic therapy to eliminate the bacteria.

The invention further provides a method of reducing the risk of a pregnant woman having an infection-associated sPTB, the method comprising the steps of:
a) testing a sample of vaginal fluid for the presence of the following bacteria:
   i) Fusobacterium nucleatum;
   ii) Ureaplasma parvum genotype SV3 and/or Ureaplasma parvum genotype SV6;
   iii) Gardnerella vaginalis; and
   iv) Lactobacillus iners
b) if either:
   Fusobacterium nucleatum in the absence of Ureaplasma parvum genotype SV3 and/or Ureaplasma parvum genotype SV6; or
   Ureaplasma parvum genotype SV3 and/or Ureaplasma parvum genotype SV6, Gardnerella vaginalis, and Lactobacillus iners
      are present, giving the pregnant woman antibiotic therapy to eliminate the bacteria.

The antibiotic therapy may optionally be followed by or administered concurrently with a probiotic therapy to reduce the chance of re-infection.

Preferably, the testing method is quantitative PCR (qPCR).

Optionally, the Gardnerella vaginalis tested for is clade 4.

Optionally, the testing is preceded by testing for the presence of high levels of Lactobacillus species other than Lactobacillus iners in the vaginal fluid, preferably Lactobacillus gasseri, Lactobacillus crispatus and/or Lactobacillus jensenii. If high levels of these Lactobacillus species are detected, then the risk of infection-associated spontaneous preterm birth (sPTB) is low and step (a) need not be carried out.

Preferably the testing is carried out at between 10 and 24 weeks' gestation, between 16 and 24 weeks' gestation, more preferably between 18 and 22 weeks' gestation, most preferably between 18 and 20 weeks' gestation, or before 22 weeks' gestation.

Preferably the antibiotic therapy is carried out at between 10 and 24 weeks' gestation, between 16 and 24 weeks' gestation, more preferably between 18 and 22 weeks' gestation, most preferably between 18 and 20 weeks' gestation, or before 22 weeks' gestation.

Preferably the probiotic therapy is carried out at between 10 and 24 weeks' gestation, between 16 and 24 weeks' gestation, more preferably between 18 and 22 weeks' gestation, most preferably between 18 and 20 weeks' gestation, or before 22 weeks' gestation.

### Kits

The present invention provides a kit to determine if a pregnant woman is at risk of an infection-associated sPTB, the kit comprising:
a) a means for testing a sample of vaginal fluid for the presence of the following bacteria:
   i) Ureaplasma parvum genotype SV3 and/or Ureaplasma parvum genotype SV6;
   ii) Gardnerella vaginalis; and
   iii) Lactobacillus iners
b) Instructions for use.

The kit may further contain a test for Fusobacterium nucleatum.

The kit may further contain a test for Lactobacillus species other than Lactobacillus iners. Preferably the Lactobacillus species other than Lactobacillus iners tested for are Lactobacillus gasseri, L. crispatus and L. jensenii.

The kit of the present invention may also include instructions designed to facilitate user compliance. Instructions, as used herein, refers to any label, insert, etc., and may be positioned on one or more surfaces of the packaging material, or the instructions may be provided on a separate sheet, or any combination thereof. For example, in an embodiment, the kit of the present invention comprises instructions for testing for the bacteria associated with sPTB of the present invention. In one embodiment, the instructions indicate that the method of the present invention is suitable for the prediction of sPTB and women who would benefit from treatment to prevent sPTB.

### General

Those skilled in the art will appreciate that the invention described herein is susceptible to variations and modifications other than those specifically described. The invention includes all such variations and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. concentration, signal, detection, amplification, sequence, etc.). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### The UPCAN Study—A Prospective Study of Ureaplasma, Mycoplasma and Candida sp. Vaginal Colonisation in Low-Risk, Asymptomatic Pregnant Women and Association with Spontaneous Preterm Birth

### Subjects

The study consisted of 206 low-risk pregnant women recruited from King Edward Memorial Hospital (KEMH), Perth, Western Australia. Fifteen cases withdrew from the study or were lost to follow-up, leaving 191 for analysis. The study was approved by the Human Research Ethics Committee of the Western Australian Department of Health, Women and Newborn Health Service (2056/EW).

### Inclusion and Exclusion Criteria

Women with a singleton pregnancy were eligible for inclusion if they were aged 18-40 years, able to speak and read English and were pregnant within the first or second trimester.

Women were excluded from the study if they were deemed to be at a high risk of PTB (one or more previous PTBs) and/or other pregnancy complications such as preeclampsia. Other exclusion criteria included current use of antifungals, tetracycline and/or macrolide antibiotics, current diagnosis of a urinary tract infection, and history of recurrent vaginal thrush.

### Questionnaires

Upon recruitment to the study and at each subsequent sampling point, women were invited to complete a de-identified medical/lifestyle questionnaire in a private setting. The questionnaire first inquired about medications currently used (antibiotic/natural/probiotic) and past diagnoses of urinary tract infections/vaginal thrush. Information regarding current and previous smoking and/or alcohol use was sought as ‘yes or no’, and subsequently followed by questions to quantify the number of cigarettes smoked/standard drinks consumed each day as appropriate. The average number of episodes of sexual intercourse per week during pregnancy was recorded.

### Pregnancy Outcome Data

Pregnancy outcome data from the hospital's electronic medical records were accessed by experienced research midwives and coded after completion of the pregnancy.

Sample Collection

Written informed consent was obtained by the attending midwife prior to enrolment in the study. This included consenting to publication of any data produced by the study. Participants provided two self-collected vaginal swabs (Copan Diagnostics, Murrieta, Calif., USA) at recruitment (median 21 wk, range 13-26 wk gestational age [GA]), ˜28 wk GA (median 29, range 24-38 wk) and ˜36 wk GA (median 36, range 32-40 wk). The first swab was employed for detection of Ureaplasma and Mycoplasma spp. and the second for detection of Candida spp. Detailed verbal, written and pictorial instructions were provided to all women in an attempt to standardise the swab collection process. Briefly, while wearing gloves, participants inserted the swab 5 cm into their vagina and gently rotated this for 20 s, ensuring the walls of the vagina came into contact with the swab. Swabs were then immediately placed into a collection tube containing either 1 mL UTM media (Ureaplasma and Mycoplasma spp.) (Copan Diagnostics) or 2 mL of CAT media (Candida spp.) (Copan Diagnostics), snapped at the mid-stem breakpoint, capped and stored at 4° C. All samples were transported to the laboratory on ice for culture within 24 h of collection.

### Detection of Ureaplasma spp.

### Culture

UTM tubes were vortexed for 10 s to release all cells from swabs. Swabs were subsequently pressed against the tube wall to release all free liquid and then discarded. 200 μL of sample was added to 1.8 mL of 10B broth (Melbourne University Media Preparation Unit) and incubated for 48 h at 37° C., 5% CO₂, 2% O₂. The remaining volume of sample was transferred to a 2 mL microfuge tube and frozen at −80° C. until DNA extraction.

Positive cultures, indicated by a pH-associated colour change (yellow>pink), were immediately transferred to 2 mL microfuge tubes and frozen at −80° C.

### DNA Extraction

DNA was extracted from 250 μL of UTM swab eluate using the Siemens Sample Preparation Kit 1.0 (Siemens, Munich, Germany) on an automated Kingfisher Duo extraction platform (Thermo Fisher Scientific Inc. Mass., USA) as per manufacturer's instructions. All extracts were eluted in a final volume of 100 μL of elution buffer (Siemens). A positive extraction control consisting of approximately 250 colour changing units (CCU) each of U. parvum and U. urealyticum was included in all runs.

### Real-Time PCR

In addition to culture, Ureaplasma spp. DNA was detected from vaginal swabs using real-time PCR. Vaginal swab DNA was screened using an assay targeting the urease gene of U. parvum and U. urealyticum, as described by Yi et al. (1), adapted for use on a ViiA7 real-time PCR system (Life Technologies, Carlsbad, Calif., USA). Reaction mixtures (final concentration) consisted of 1×Taqman FAST Advanced Master Mix (Life Technologies), 0.9 μM primers UU1613F and UU1524R (Life Technologies), 0.25 μM probes UU-parvo (FAM) and UU-T960 (VIC) (Life Technologies), 5 μL of template DNA and nuclease-free water (Ambion, Life Technologies) to a final volume of 20 μL. PCR cycling conditions consisted of an initial denaturation/Taq activation at 95° C. for 20 s, followed by 40 quantification cycles of 95° C. for 1 s and 60° C. for 20 s (data acquiring). Positive standards were included in each run.

### High-Resolution Melt PCR

Samples that were positive for U. parvum DNA were genotyped and classified as either serovar (SV) one, SV3, SV6 or SV14 using our previously described high resolution melt (HRM) PCR assay targeting the multiple-banded antigen gene (2) on a ViiA7 real-time PCR system (Life Technologies). Reaction mixtures (final concentration) consisted of 1×Amplitaq Gold 360 buffer (Life Technologies), 1.5 mM MgCl₂ (Life Technologies), 200 μM of each dNTP (Life Technologies), 0.3 μM primers UPHRM-F and UPHRM-R (Life Technologies), 1×MeltDoctor HRM dye (Life Technologies), Amplitaq Gold 360 DNA polymerase (0.1 U/μL) (Life Technologies), 10 μL of template DNA and nuclease-free water (Ambion, Life Technologies) to a final volume of 20 μL. PCR cycling conditions consisted of an initial denaturation/Taq activation at 95° C. for 10 min, followed by 40 cycles of 95° C. for 15 s and 60° C. for 1 min (data acquiring). To provide data on U. parvum serovar status, amplicons were subsequently subject to a HRM step where the temperature was raised to 95° C. for 10 s and then lowered to 60° C. for 1 min. The temperature was then raised to 95° C. at a rate of 0.025° C./s (continuous data acquisition), held at 95° C. for 15 s and then lowered to 60° C. for 15 s. HRM profiles were analysed using ViiA7 real-time PCR system software v1.2.1 (Life Technologies). All samples were run in duplicate and positive standards of U. parvum SV1, SV3, SV6 and SV14 were included in each run.

### Sequencing

Following HRM analysis, samples that produced non-standard melt curve patterns were subject to DNA sequencing. PCR amplicons were generated using the same HRM primer set on a Veriti PCR thermocycler (Life Technologies). Reaction mixtures (final concentration) consisted of 1×Amplitaq Gold 360 buffer (Life Technologies), 2.0 mM MgCl₂ (Life Technologies), 200 μM of each dNTP (Life Technologies), 0.5 μM primers UPHRM-F and UPHRM-R (Life Technologies), Amplitaq Gold 360 DNA polymerase (1.25U) (Life Technologies), 5 μL of template DNA and nuclease-free water (Ambion, Life Technologies) to a final volume of 50 μL. PCR cycling conditions consisted of an initial denaturation/Taq activation at 95° C. for 10 min, followed by 40 cycles of 95° C. for 15 s, 56° C. for 30 s and 72° C. for 45 s. A final extension step of 72° C. for 7 min was also included.

PCR amplicons were checked for size (305 bp) on a 1.5% agarose gel stained with Gel Red (Biotium) and subsequently purified using a QIAquick PCR purification kit (QIAGEN) as per manufacturer's instructions. Purified DNA fragments were sequenced using Big Dye version 3.1 chemistry (Applied Biosystems) and post-cleaned using SPRI. Fragments were separated on a 3730xl DNA Analyser using a 96-capillary array (Applied Biosystems) at the Australian Genome Research Facility (Perth, Western Australia).

### Detection of Mycoplasma spp.

### DNA Extraction

DNA was extracted from 250 μL of UTM swab eluate as described above.

### Real-Time PCR

Mycoplasma hominis

M. hominis DNA was detected in vaginal swabs using real-time PCR. DNA samples were screened using an assay targeting the yidC gene of M. hominis as described by Ferandon et al. (3), adapted for use on a ViiA7 real-time PCR system (Life Technologies). Reaction mixtures (final concentration) consisted of 1×Taqman FAST Advanced Master Mix (Life Technologies), 0.9 μM primers MHyidCfwd and MHyidCrev (Life Technologies), 0.25 μM probe MHyidC (FAM) (Life Technologies), 5 μL of template DNA and nuclease-free water (Ambion, Life Technologies) to a final volume of 20 μL. PCR cycling conditions were as described for Ureaplasma spp. A positive standard was included in each run.

Mycoplasma genitalium

M. genitalium DNA was detected in vaginal swabs using real-time PCR. DNA samples were screened using an assay targeting the MgPa gene of M. genitalium as described by Jensen et al. (4), adapted for use on a ViiA7 real-time PCR system (Life Technologies). Reaction mixtures (final concentration) consisted of 1×Taqman FAST Advanced Master Mix (Life Technologies), 0.9 μM primers MgPa-355F and MgPa-432R (Life Technologies), 0.25 μM probe MgPa-380 (VIC) (Life Technologies), 7.9 μL of template DNA and nuclease-free water (Ambion, Life Technologies) to a final volume of 20 μL. PCR cycling conditions consisted of an initial denaturation/Taq activation at 95° C. for 20 s, followed by 50 quantification cycles of 95° C. for 1 s and 60° C. for 20 s (data acquiring). Positive standards were included in each run.

### Detection of Candida spp.

### Culture

CAT tubes were vortexed for 10 s to release all cells from swabs. Swabs were subsequently pressed against the tube wall to release all free liquid and then disposed of. 1 mL of sample was transferred to a 2 mL microfuge tube and frozen at −80° C. until DNA extraction. The remaining sample (approximately 900 μL) was incubated at 37° C. for 24 h to enrich for low cell titres of Candida spp. Following incubation, two 10 μL loops of sample were plated onto Candida Brilliance agar (Oxoid, Thebarton, South Australia, Australia) and incubated at 37° C. for 72 h.

Positive cultures on Candida Brilliance agar (Oxoid) were classified as follows: Green colonies=C. albicans; pink/yellow/beige/brown colonies=non-albicans Candida spp. All positive cultures were re-plated for purity and following incubation, pure cultures were re-suspended in 2 mL of Sabaraud-Dextrose broth (Oxoid) and frozen at −80° C.

### DNA Extraction

DNA was extracted from 250 μL of pure Candida sp. isolate broth resuspension as described above.

### Real-Time PCR

To confirm the identification of non-albicans Candida spp. isolated using Candida Brilliance agar, a multiplex real-time PCR assay targeting the RNase P RNA (RPR) gene of Candida sp. and C. glabrata was used. Primer and probe designs were similar to that of Innings et al. (5), but were optimised for use on a ViiA7 real-time PCR system (Life Technologies). Reaction mixtures (final concentration) consisted of 1×Taqman FAST Advanced Master Mix (Life Technologies), 0.9 μM primers CAND-CR1F (5′ CGGGTGGGAAATTCGGT 3′), CAND-CR5R (5′ CAATGATCGGTATCGGGT 3′), GLA-F (5′ TGGCTCACACACTTTGTCACTTT 3′) and GLAR (5′ ACCTCGCCTCACACCAATG 3′) (Life Technologies), 0.25 μM probes ALLCAN (NED-TTCGCATATTGCACTMAAYAGC-MGB) and GLA (VIC-AACCTGCCATTTCCGCTCCCTTAAGA-TAMRA) (Life Technologies), 5 μL of template DNA and nuclease-free water (Ambion, Life Technologies) to a final volume of 20 μL. PCR cycling conditions were as described above for Ureaplasma spp.

### Statistical Analyses

Data were summarised using frequency distributions for categorical data, and median, interquartile range and range for continuous data. Categorical outcomes were compared using Chi-square and Fisher's exact tests, and continuous outcomes compared using Mann-Whitney tests. All analyses were conducted on detection of microbes at recruitment, due to the relatively stable colonisation levels throughout pregnancy. SPSS Version 20.0 (Armonk, N.Y.: IBM Corp) statistical software was used for data analysis. P-values<0.05 were considered statistically significant.

### Results

206 women in total were recruited to the study. From these, 15 withdrew or were lost to follow-up. Demographic/birth and lifestyle characteristics of the 191 women that formed the final study cohort are provided below (Table 1). The overall PTB rate (<37 wk GA) was 9%, which included 13 spontaneous births and four births that required labour induction or caesarean delivery for maternal or fetal indications. These four births were excluded in the comparison of microorganisms between preterm and term births. There were six births (five spontaneous) <34 wk GA (3%) and two births with a birth weight <1500 g (1%).

TABLE 1
Demographic, birth and lifestyle characteristics
of women in the study

| | |
|---|---|
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |

y years.
GA gestational age.
wk weeks.
g grams [symbol]
Data represents median (interquartile range:range) or N (%). as appropriate [symbol]
indicates data missing or illegible when filed

Detection of Vaginal Ureaplasma, Mycoplasma and Candida spp. during Pregnancy

Vaginal detection rates for Ureaplasma spp., Mycoplasma spp. and Candida spp. varied substantially at both the genus and species level (Table 2). Ureaplasma spp. were the most common of the three organisms detected, present in 44-48% of women over the three sampling points. Within this genus, U. parvum was the most common species detected, 3-4 times more prevalent than U. urealyticum (Table 2).

Candida spp. were the second most common organism detected, present in 34-38% of women. Within this genus, C. albicans was by far the most common species detected, 6-25 and 10-24 times more prevalent than C. glabrata and non-albicans/non-glabrata Candida spp., respectively (Table 2).

Detection rates for M. hominis and M. genitalium were much lower than for Ureaplasma spp. and Candida spp. M. hominis detection rates ranged from 8-11% over the three sampling points, whilst for M. genitalium, rates ranged from 2-3% (Table 2).

TABLE 2
Detection rates for vaginal Ureaplasma, Mycoplasma
and Candida spp. during pregnancy

| | [symbol] | [symbol] | [symbol] |
|---|---|---|---|
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |

^{a}Due to variations in sample compliance, apparent reductions or increases in genotypes over the three time points are not indicative of genotype stability
^{b}represents median, range
^{c}Same study participants [symbol]
indicates data missing or illegible when filed

### Comparison of Culture and Real-Time PCR for Detection of Ureaplasma spp.

Detection rates of Ureaplasma spp. by 10B broth culture at 37° C. (5% CO₂, 95% N₂) were almost identical to detection rates by real-time PCR. Concordance between the two techniques was 99, 100 and 100% over the three time-points, respectively.

High-Resolution Melt PCR Genotyping of U. parvum

HRM PCR was able to resolve singular U. parvum genotypes in 91% of colonised clinical samples. U. parvum genotype SV6 was the most common detected, closely followed by SV3, SV1 and SV6.1, respectively. No cases of genotype SV14 were found (Table 2). An additional 3% of cases were resolved to the ‘mixed’ genotype level, suggesting the presence of two or more U. parvum genotypes within the same sample. There was also a small number of cases where either amplification was too weak to produce a melt curve sufficient for genotype discrimination (1%) or no amplification was produced whatsoever (3%). In addition, for one study participant, all three samples produced slightly different HRM curves, which upon DNA sequencing, showed four unique nucleotide polymorphisms within the targeted region of the multiple-banded antigen gene, not indicative of any of the four characterised U. parvum genotypes. The sequence was most closely matched to genotype SV6, and as a result was deemed genotype SV6.1.

### Vaginal Colonisation Dynamics

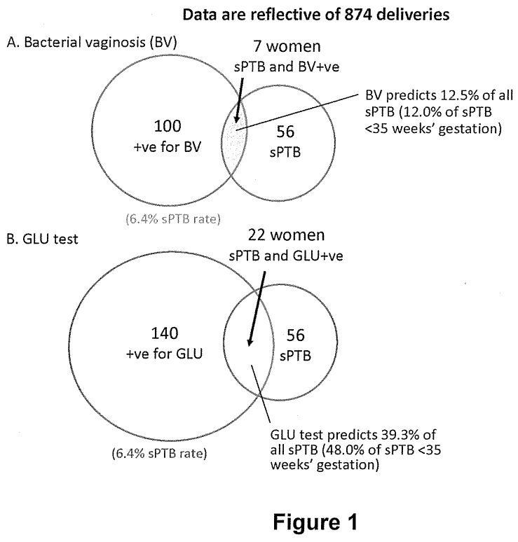

Of the 191 study participants, 134 provided samples at all three time points. In these women, detection rates for all organisms showed minimal variance over the duration of the study (FIG. 1). M. genitalium (1.5% at all three points) had the lowest variance of all organisms detected, followed by U. parvum (36.6-37.3%), U. urealyticum (9.7-11.2%) and non-albicans/non-glabrata Candida spp. (0.7-2.2%), C. albicans (32.1-34.3%) and M. hominis (9-11.2%), and last, C. glabrata (1.5-5.2%). These results are inclusive of one case where U. parvum/U. urealyticum was detected at recruitment sampling, after which U. urealyticum was not detected again in subsequent samples.

Similarly, there was very little variance in U. parvum genotype detection at any of the three time points. In nearly all cases, the genotype detected at recruitment was maintained throughout the second and third time points. The only exceptions were in one case where a participant colonised by U. parvum genotype SV6 at recruitment showed a mixed genotype profile at the later time points, and in another case where mixed genotype profiles were detected at all three time points. This made it impossible to be certain that the same combination of genotypes was present in each instance, due to the limitations of the HRM assay. There were also an additional two instances where the HRM assay failed to generate sufficient amplification of the second sample to allow accurate genotype discrimination. However, in both of these cases, the first and third sample genotype identifications were concordant.

Association between Pharmaceutical & Lifestyle Factors and Detection of Organisms at Recruitment

Based upon answers provided by 189 study participants at recruitment (Table 3), Ureaplasma spp. and Mycoplasma spp. were detected more frequently in women who previously smoked (Ureaplasma spp.—37% present vs. 17% absent, p=0.002; and Mycoplasma spp.—44% present vs. 24% absent, p=0.036), and in women who had sexual intercourse≥3 times per week during pregnancy (Ureaplasma spp.—35% present vs. 18% absent, p=0.018; and Mycoplasma spp.—56% present vs. 21% absent, p=0.001).

Candida spp. were detected more frequently in women who continued to smoke during their pregnancy (Candida spp.—18% present vs. 7% absent, p=0.020).

TABLE 3
Associate between pharmaceutical/lifestyle characteristics and detection of Ureaplasma,
Mycoplasma and Candida spp. at recruitment (n = 189)

| | Ureaplasma spp. | | | Candida spp. | | | Mycoplasma spp. | | |
|---|---|---|---|---|---|---|---|---|---|
| | Yes | No | | Yes | No | | Yes | No | |
| Question | (n = 89) | (n = 100) | p-value | (n = 73) | (n = 116) | p-value | (n = 25) | (n = 164) | p-value |
| Current antibiotic, antifungal | 1 (1%) | 7 (7%) | 0.045 | 3 (4%) | 5 (4%) | 0.947 | 1 (4%) | 7 (4%) | 1.000 |
| or probiotic use? | | | | | | | | | |
| Ever diagnosed with a UTI? | 32 (36%) | 43 (43%) | 0.296 | 30 (41%) | 45 (39%) | 0.789 | 8 (32%) | 67 (41%) | 0.387 |
| Ever diagnosed with thrush? | 40 (46%) | 51 (51%) | 0.448 | 34 (47%) | 57 (49%) | 0.798 | 10 (42%) | 81 (49%) | 0.479 |
| Current smoker? | 13 (15%) | 8 (8%) | 0.149 | 13 (18%) | 8 (7%) | 0.020 | 5 (20%) | 16 (10%) | 0.165 |
| Previous smoker? | 33 (37%) | 17 (17%) | 0.002 | 22 (31%) | 28 (24%) | 0.351 | 11 (44%) | 39 (24%) | 0.036 |
| Currently consumes alcohol? | 1 (1%) | 3 (3%) | 0.624 | 2 (3%) | 2 (2%) | 0.642 | 1 (4%) | 3 (2%) | 0.438 |
| Previously consumed alcohol? | 48 (54%) | 60 (61%) | 0.355 | 39 (53%) | 69 (60%) | 0.374 | 14 (56%) | 94 (58%) | 0.875 |
| Current frequency of sexual | | | | | | | | | |
| intercourse? | | | | | | | | | |
| <1 week | 20 (23%) | 20 (23%) | 0.018 | 14 (20%) | 28 (24%) | 0.715 | 3 (12%) | 39 (24%) | 0.001 |
| 1-2 times/week | 38 (43%) | 58 (60%) | | 37 (52%) | 59 (51%) | | 8 (32%) | 88 (55%) | |
| ≥3 times/week | 31 (35%) | 17 (18%) | | 20 (28%) | 28 (24%) | | 14 (56%) | 34 (21%) | |

^{a}wk week; bold type indicates statistical significance (p < 0.05)

Association between Vaginal Microbial Colonisation and Spontaneous Preterm Birth Spontaneous Preterm Birth <37 Wk GA

The overall microbial characteristics of vaginal samples collected during the study are provided in Table 4. Ureaplasma spp. were detected more frequently [85% (95% CI: 62-100%) vs. 45% (37-52%), p=0.006] in the recruitment samples of women who delivered preterm compared to those who delivered at term (Table 4). At the species level, the presence of U. parvum was significantly increased among PTB cases [77% (50-100%) vs. 36% (29-43%), p=0.004]. There was a small, but significant association between the titre of U. parvum and PTB, with an average U. parvum titre of 10⁶ CCU in cases of PTB vs. 10⁵ CCU for term cases. U. parvum genotypes SV3 and SV6 were equally represented amongst term pregnancies; however, in women who delivered preterm, genotype SV6 was significantly more common, present in 54% (22-85%) of preterm deliveries compared to 15% (10-20%) of term deliveries (p=0.002). When detected alone, the presence of Candida spp. was not associated with PTB at either the genus or species level. However, when C. albicans was detected alongside U. parvum a significant positive association with PTB was observed [46% (15-78%) vs. 13% (8-18%), p=0.005]. This association strengthened when U. parvum genotype SV6 was present [39% (8-69%) vs. 7% (3-11%), p=0.003].

There was no apparent association between presence of Mycoplasma spp. and PTB; however, M. genitalium was more common in the recruitment samples from women who delivered preterm vs. at term (15 vs. 2%, respectively) and this result trended towards significance (p=0.057). Similarly, Candida spp. were also more common in the recruitment samples from women who delivered preterm vs. at term (54 vs. 36%, respectively); however, this difference was not statistically significant (p=0.241).

No association was detected between PTB and the presence of either U. urealyticum, C. albicans, C. glabrata, non-albicans/glabrata Candida spp. or M. hominis.

TABLE 4
Vaginal colonisation rates of Ureaplasma, Mycoplasma
and Candida spp. at recruitment in women who
delivered spontaneously pre-term vs. at term

| | [symbol] | [symbol] | |
|---|---|---|---|
| | [symbol] | [symbol] | [symbol] |
| Ureaplasma spp. | 11 (85%) | 78 (45%) | 0.006 |
| U. parvum | 10 (77%) | 63 (36%) | 0.004 |
| [symbol] | [symbol] | [symbol] | [symbol] |
| Titre (CCU) of | 6 (5-6; 3-7) | 5 (4-6; 2-8) | 0.043 |
| U. parvum | | | |
| Serovar 3 of U. parvum | 3 (23%) | 23 (13%) | 0.399 |
| Serovar 6 of U. parvum | 7 (54%) | 26 (15%) | 0.002 |
| Candida spp. | 7 (54%) | 63 (23%) | 0.241 |
| C. albicans | 6 (46%) | 55 (32%) | 0.358 |
| C. glabrata | 1 (8%) | 9 (5%) | 0.522 |
| Non albicans/non glabrata | 0 | 4 (2%) | 1.000 |
| Mycoplasma spp. | | | |
| [symbol] | 2 (15%) | 19 ( [symbol] %) | 0.644 |
| M. genitallum | 2 (15%) | 4 (2%) | 0.057 |
| U. parvum + C. albicans | 6 (46%) | 22 (13%) | 0.005 |
| U. parvum serovar 6 + | 5 (39%) | 12 (7%) | 0.003 |
| C. albicans | | | |

^{a}Bold type indicates statistical significance (p <0.05)
^{b}median, interquartile range, range [symbol]
indicates data missing or illegible when filed

### Spontaneous Preterm Birth <34 Wk GA and Birthweight <1500 g

Five babies were born <34 wk GA, including two weighing <1500 g (Table 5). U. parvum was detected at recruitment in all cases and in 80% (4/5) of these, genotype SV6 was present. In the four earliest preterm deliveries (25.9-31.4 wk GA), both U. parvum and C. albicans were detected at recruitment (Table 5). Unfortunately, due to insufficient numbers, no statistical analyses were able to be performed on risk of PTB <34 wk GA.

TABLE 5
Detection of organisms at recruitment and birth characteristics for babies
born spontaneously at <34 weeks GA (n = 5)

| GA (WK) | BW (g) | Sex | U. parvum | [symbol] (CCU-10 [symbol] ) | U. parvum/U. urealyticum | U. parvum SV6 | C. albicans | M. hominis | M. genitalium |
|---|---|---|---|---|---|---|---|---|---|
| 25.9 | 690 | M | ✓ | 6 | U. parvum | ✓ | ✓ | ✓ | ✓ |
| 28.0 | 760 | M | ✓ | 7 | U. parvum | ✓ | ✓ | | |
| 30.9 | [symbol] 560 | M | ✓ | 6 | U. parvum | | ✓ | ✓ | |
| 31.4 | [symbol] 950 | M | ✓ | 5 | Both | ✓ | ✓ | | ✓ |
| 32.0 | [symbol] 820 | — | ✓ | 6 | U. parvum | ✓ | | | |

^{a}wk weeks, g grams, CCU colour changing units; a tick (✓) represents presence of organism, a dash (—) absence of organism [symbol]
indicates data missing or illegible when filed

### Example 2

### The Predict1000 Study—Microbial Biomarkers for the Prevention of Preterm Birth

Two studies will be conducted: A large cohort study of women presenting for antenatal care and a smaller sub-study of novel microbial biomarkers.

### Cohort Study

This study will expand the data on prevalence rates of U. parvum, U. urealyticum and M. hominis within the vagina during pregnancy, and will extend this further by defining the prevalence of key organisms associated with BV within this cohort.

In addition, the study will document vaginal fluid pH and sialidase levels during pregnancy and will be suitably powered to detect associations between all of these factors and the primary and secondary outcomes. Both nulliparous and multiparous women attending antenatal clinics at KEMH before 20 weeks' gestation over a 12 month period will be invited to participate. Recruitment will be enriched by preferential selection of women with a history of prior PTB. Women will be ineligible if they are taking antibiotics or antimycotics, have a multiple pregnancy, have a cervical suture or are using vaginal progesterone.

### Primary & Secondary Endpoints

The primary end point is PTB before 37 weeks' gestation

Among the secondary endpoints are: PTB before 34 weeks' gestation; threatened preterm labour at any gestational age; PPROM; low birth weight; very low birthweight; neonatal sepsis or other morbidities; clinical and/or histologic chorioamnionitis.

Participation will involve:
i) Completion of a questionnaire inquiring about lifestyle, diet, sexual activity, infections (current/previous) and any antibiotic/probiotic use within the previous 12 months.
ii) A speculum-assisted swab from the vaginal posterior fornix collected by a research midwife for microbial DNA analysis (qPCR) and sialidase measurement (fluorescent substrate cleavage assay).
iii) A second swab for microbial culture (Ureaplasma spp. and M. hominis).
iv) An assessment of vaginal fluid pH.
v) Collection of placentas from all births <34 weeks' gestation consistent with Hospital policy.

### Novel Microbial Biomarkers Sub-Study

This sub-study will involve metagenomic analysis of vaginal swabs from all women in the cohort study who delivered prior to 34 weeks' gestation following spontaneous PTL or PPROM, matched with a similar number of women who delivered at term by Caesarean section without complications. We estimate 50 cases matched with 50 controls. This sub-study will provide genus and species-level data regarding the microbial community composition of the vagina during pregnancy. It will also compare the vaginal microbial communities of preterm and term pregnancies, identifying microbial genera and species associated with risk of PTB and greatly enhancing the diagnostic sensitivity of the risk-scoring system.

Placentas from all deliveries less than or equal to 34 weeks' gestation will be collected for histological examination and microbiological culture as per routine clinical practice. In addition, sub-amniotic swabs will be taken from four sites across the placental plate to sample any microbiota associated with intraamniotic infection (free of maternal vaginal contamination). The vaginal and placental swabs will be retrospectively analysed using a 16S rRNA gene metagenomic approach in order to confirm the vagina as the source of intraamniotic infection through microbial community comparison.

### Sample Collection and Need for Treatment

All pregnant women attending KEMH are routinely screened for Chlamydia trachomatis and Neisseria gonorrhoeae by urine testing and treated accordingly. In addition, any symptoms suggestive of a vaginal infection, such as Candida spp. thrush, are managed by the taking of appropriate samples and prescription of treatment. Vaginal swabs collected during the research study will be cultured using our current Ureaplasma spp. and M. hominis culture protocols.

The culture component of this study will be primarily used to collect a catalogue of isolates for future strain-specific analyses. In addition to culture, both of these organisms, along with the BV-associated organisms, G. vaginalis, A. vaginae, Megasphaera spp. and Bacterial Vaginosis-Associated Bacteria-2 (BVAB-2) will be detected and semi-quantitated by real-time PCR analyses. Candida spp. will be detected using real-time PCR analyses also, as previously described by CIC Payne (P011679345).

Vaginal fluid pH and sialidase levels will also be measured by pH glove and fluorescence substrate assay, respectively.

### Sample Collection and Analysis

### Vaginal Swabs

Two swab collection kits will be used during the study. The first of these is a clinically-validated Ureaplasma/Mycoplasma-specific, Universal Transport Medium (UTM) kit (Copan Diagnostics). UTM kits contain a flocked swab and a vial of UTM designed to support the growth of Ureaplasma/Mycoplasma spp. In addition to this, a highly flocked swab (Copan Diagnostics) will be used for the collection of all samples for molecular analyses.

### Culture Analysis

Following collection, swabs will be immediately placed into UTM by the midwife, capped and stored at 4° C. for a maximum of 24 h prior to processing. 200 μL of sample will be added to 1.8 mL of 10B media containing urea (Ureaplasma spp.) and 10B media containing arginine (M. hominis) and incubated at 37° C./48-120 h. Positive cultures will be purified using a broth micro-dilution method and 1 mL of culture will be frozen at −80° C. for future genotypic analyses.

### PCR Analysis

Flocked vaginal swabs will placed back in the collection tube and immediately stored at 4° C. for <24 h prior to processing. Swabs will be thoroughly resuspended in 2 mL PBS; a 100 μL aliquot will be removed for measurement of vaginal sialidase levels and the remaining volume of eluate will be centrifuged at 20,000×g, 4° C. for 20 min and the supernatant removed. Pellets will be resuspended in 250 μL of PBS and DNA extracted from the entire volume using a Stratec InviMag Universal Bacteria kit (ThermoFisher) on a Kingfisher extraction platform.

Semi-quantitative, real-time PCR assays will be used to detect U. parvum and U. urealyticum (1), M. hominis (2), sialidase positive and negative G. vaginalis (3), A. vaginae, Megasphaera spp. and BVAB-2 (4) on an Applied Biosystems ViiA7 real-time PCR system. All U. parvum positive swabs will be further analysed using a high-resolution melt PCR genotyping assay (5) to document cases of individual and mixed serovar colonisations.

### Metagenomics

DNA extraction from vaginal and placental swabs will be conducted as described above. For all extracts, following confirmation of presence of bacterial DNA by PCR, the entire 16S rRNA gene will be amplified using the 8F/1492R primer set (6) (1.5 kB full size) and positive amplicons purified with a QIAGEN PCR purification kit. Purified PCR amplicons from individual samples will have sequencing adaptors and barcodes attached and undergo sequencing on a PacBio Sequel next-generation sequencing platform. Sequence data will be processed using the Quantitative Insights into Microbial Ecology (QIIME) software package (7). For phylogenetic information, sequence homologies of 97% and 99% will be used for genus and species identification respectively.

### Placental Histopathology and Microbiological Analysis

Placentas from all cases of PTB≥34 weeks' gestation will be transported to the Histopathology Department for histopathological examination and microbiology as part of routine clinical management. We expect to have ˜50 cases of birth <34 weeks' gestation. An equal number of normal term placentas delivered by Caesarean section will serve as controls.

Histopathological examination will be performed by an experienced perinatal pathologist blinded to the clinical outcomes. Semi-quantitative histologic scoring of the extraplacental membranes, umbilical cord, chorionic plate and placenta will be conducted using our standard scoring system.

All placentas collected will be cultured as described previously. The sub-amniotic swab is cultured for aerobic organisms in addition to Haemophilus influenzae.

A portion of this sample will be transported to the research laboratories for extraction of microbial DNA for metagenomic analysis for comparison with the corresponding vaginal sample.

### Lifestyle and Clinical Data Collection

Maternal questionnaires will inquire about symptoms of vaginal discharge or irritation, dysuria, recent/past urinary tract infection or vaginal infection, smoking practices, frequency/nature of sexual intercourse and antibiotic/probiotic use. Obstetric and neonatal outcome data from all women in the study will be obtained from hospital databases.

### Statistical Power

The overall PTB rate at KEMH is 25% and the state-wide prevalence is 8.8%; we anticipate that recruitment in our clinics with enrichment of high-risk cases will result in a PTB rate of at least 15%. The sample size of 1000 women used in logistic regression analysis in which we will model risk of PTB will attain at least 90% power to detect the effects of microbial colonisation exceeding a two-fold increase in PTB risk (equivalent to odds ratios≥2.50) when the PTB rate is at least 12.0% (i.e. from 12.0 to 25.4% or 15.0 to 30.6%), while simultaneously adjusting for other relevant microbiological findings and clinical risk factors with partial r²=0.1

### Statistical Analysis

The prevalence of colonisation with genital mycoplasmas, the distinct U. parvum serovars and BV-related microflora will be estimated using binomial distribution. Primary statistical analysis will generate predictive scores for bacterial infection-related PTB to identify women at high risk, using microbial profile in early pregnancy alone and microbial profile adjusted for other relevant maternal and obstetric characteristics. Univariable and multivariable logistic regression analysis will be used to construct microbial and adjusted microbial risk scores of all primary and secondary clinical endpoints. Logistic regression analyses to derive these microbial predictive risk scores of PTB will be supplemented with recursive partitioning models, such as binary, regression and survival trees, designed to explore the non-linear relationships within the microbial profiles and with other obstetrics risk factors before performing logistic regression analysis. Secondary evaluation of the magnitude of the effect of the microbial profiles on the gestational age at delivery and at gestational ages when secondary clinical endpoints occur will be conducted using proportional Cox regression. Comparisons between the vaginal/placental microbial profiles within preterm and term pregnancies will be undertaken using principal component analyses.

Some data has been collated from the Predict1000 study. Table 6 shows the presence/absence of all bacteria screened for in this study in relation to sPTB, nsPTB and term deliveries.

TABLE 6
Presence /Absence of Bacterial species in Predict1000 study.

| | | GA weeks at birth | | | | | |
|---|---|---|---|---|---|---|---|
| | | sPTB | | nsPTB | | Term | |
| | | N | (%) | N | (%) | N | (%) |
| UP3 and/or UP6 | No | 31 | 55.4% | 34 | 54.8% | 510 | 62.3% |
| | Yes | 25 | 44.6% | 28 | 45.2% | 308 | 37.7% |
| c_GassCrispJens_or01 | No | 48 | 85.7% | 40 | 66.7% | 563 | 68.8% |
| | Yes | 8 | 14.3% | 20 | 33.3% | 255 | 31.2% |
| c_Llners | No | 26 | 46.4% | 27 | 44.3% | 402 | 49.1% |
| | Yes | 30 | 53.6% | 34 | 55.7% | 416 | 50.9% |
| c_Gvag | No | 26 | 46.4% | 27 | 43.5% | 380 | 46.5% |
| | Yes | 30 | 53.6% | 35 | 56.5% | 438 | 53.5% |
| c_Fnucl | No | 41 | 73.2% | 45 | 72.6% | 656 | 80.2% |
| | Yes | 15 | 26.8% | 17 | 27.4% | 162 | 19.8% |
| c_Pepto | No | 41 | 73.2% | 35 | 56.5% | 599 | 73.2% |
| | Yes | 15 | 26.8% | 27 | 43.5% | 219 | 26.8% |
| c_CAlb | No | 49 | 87.5% | 52 | 83.9% | 686 | 83.9% |
| | Yes | 7 | 12.5% | 10 | 16.1% | 132 | 16.1% |
| c_AVag | No | 19 | 33.9% | 15 | 24.2% | 202 | 24.7% |
| | Yes | 37 | 66.1% | 47 | 75.8% | 616 | 75.3% |
| c_Sialid | No | 37 | 66.1% | 36 | 58.1% | 512 | 62.6% |
| | Yes | 19 | 33.9% | 26 | 41.9% | 306 | 37.4% |
| c_Lamni | No | 50 | 89.3% | 55 | 88.7% | 736 | 90.0% |
| | Yes | 6 | 10.7% | 7 | 11.3% | 82 | 10.0% |
| c_Ssang | No | 54 | 96.4% | 57 | 91.9% | 770 | 94.1% |
| | Yes | 2 | 3.6% | 5 | 8.1% | 48 | 5.9% |
| c_Mindo | No | 55 | 98.2% | 60 | 96.8% | 806 | 98.5% |
| | Yes | 1 | 1.8% | 2 | 3.2% | 12 | 1.5% |
| c_Mhomi | No | 54 | 96.4% | 55 | 88.7% | 798 | 97.6% |
| | Yes | 2 | 3.6% | 7 | 11.3% | 20 | 2.4% |
| c_Mgenit | No | 56 | 100.0% | 61 | 98.4% | 816 | 99.8% |
| | Yes | 0 | .0% | 1 | 1.6% | 2 | .2% |
| c_Prevo | No | 56 | 100.0% | 62 | 100.0% | 818 | 100.0% |
| c_Mobil | No | 56 | 100.0% | 62 | 100.0% | 818 | 100.0% |

### Example 3

### Clinical Trial of a “Screen and Treat” Program

### Trial Design

A prospective, open-label, randomized clinical trial of a novel maternal microbiological “screen & treat” program for the prevention of preterm birth.

Mid-pregnancy identification of unselected women with singleton pregnancies and vaginal microbial profiles associated with increased risk of PTB, followed by targeted antimicrobial treatment, will reduce the rate of spontaneous preterm deliveries by at least 30%.

### Inclusion and Exclusion Criteria

Women will be eligible for inclusion if they have a singleton pregnancy, ≥16 years old and ultrasound-confirmed GA.

Women will be ineligible for inclusion if they have multiple pregnancies, symptomatic vaginal infections, vaginal bleeding, rupture of membranes, active contractions, antimicrobial therapy≤14 days prior to recruitment.

### Primary & Secondary Endpoints

The primary endpoint is a ≥30% reduction in sPTB ≤37 weeks in the intervention vs. control group.

Among the secondary endpoints are Core Outcome Measures including sPTB≤34 and 28 weeks, miscarriage, birthweight ≤2500 g and ≤1500 g, iatrogenic PTB, sPTB in GLU+ve women; PPROM, preeclampsia, treatment response, maternal mortality & sepsis, neonatal mortality, composite neonatal morbidity, NICU admission/duration, neonatal sepsis (late or early), IUGR, and histological chorioamnionitis.

### Recruitment

Women attending antenatal clinics at 18-20 weeks' gestation across three WA maternity hospitals (KEMH, Osborne Park Hospital and SJOG-Midland Hospital) will be recruited. After informed consent has been obtained and study ID number assigned, baseline demographic data will be collected. Women will self-collect a pre-labelled vaginal COPAN E-swab (containing a stabilisation fluid that preserves microbial integrity for 24 h at room temperature) which will be placed into a collection box for daily collection and transport to a centralised laboratory for −80° C. storage and subsequent extraction and analysis.

### Randomization & Blinding

Randomization to either the intervention or control arm will be performed by a customized randomisation program that will randomly allocate treatment group while stratifying by nulliparity, history of PTB and study site (1:1 allocation ratio). The group allocations will be performed at the Women & Infants Research Foundation (WIRF) Trial Coordination Office. Allocation bias will be avoided by randomizing participants blind to the GLU testing procedure. All swabs will be processed and screened according to the same protocol; sample processing and result notification will take place within 4 working days of sample collection.

For participants in the control group, the result of the screening test will not be revealed to participants until after the study and they will continue to receive normal maternity care (including treatment if symptoms of vaginal infection appear). They will be not notified of their allocation until after the recruitment and delivery stage of the study has been completed. A placebo will not be used as a) the placebo might itself impact upon vaginal microbiota and dysbiosis; b) knowledge of colonisation status might alter behaviour of participants; and c) this would depart from normal obstetric care, which is the primary comparator in this trial.

Women in the intervention group will be notified of their group allocation and screen status (positive or negative) approximately one week after recruitment. For those who screen positive, the results of the test will be sent to their recruiting midwife; they will then contact them with this information and a recommended treatment plan (see below). Participants will then be mailed a pack containing medications tailored according to the result of the screening test. Women in the intervention group will not be blinded to allocation as they will need to be informed of their status so that treatment can be provided.

Delivery outcome data will be obtained from hospital and private medical records.

### Screening Test

Swabs will be analysed by multiplex GLU PCR assay. Extraction of DNA and analysis will be performed in a molecular microbiology laboratory using automated technologies to achieve optimal efficiency, accuracy and turn-around times. DNA will also be stored for in-depth microbiome analyses in follow-up studies to explore improvements in risk prediction and response to treatment.

### Intervention

Women who are screened GLU+ve will receive oral azithromycin (250 mg for 7 days) and vaginal clindamycin cream (2%) for 7 days. These are standard antibiotic regimens and widely used in pregnancy. Immediately following antimicrobial treatment, women will commence vaginal probiotic therapy with Canesflor (Bayer). Treatment consists of one vaginal capsule each night for six consecutive days, followed by one capsule per week for four weeks.

Participants will be contacted by phone/text by study research midwives a few days after the medications are mailed out to ensure participants have obtained their prescription and check comprehension and compliance. Women in the intervention group will be asked to re-take their swabs at 26-28 weeks (after completion of the 5-week probiotic course) and mail them on the day of collection using a pre-addressed express post envelope to the lab for re-testing. The results will then be relayed to them via the research midwives. A questionnaire on medication compliance and feedback will also be returned at this time. Based on published data on the efficacy of antimicrobial treatment of BV plus probiotic therapy, we expect treatment success to exceed 90%.

### Statistical Power

Based on the WA 2015 singleton pregnancy PTB rate of 6.9%, assuming 70% of these are sPTBs (9) (4.83% of all births), recruitment of 3087 women per group (6174 overall) will attain 80% power to detect a 30% reduction in the rate of sPTB in the intervention group—from 4.83% to 3.38%—when using a two-sided z-test of proportion at [symbol] =0.05; ˜494 women per group are expected to screen positive. This sample size also allows for a single interim analysis with the O'Brien-Fleming spending function used to determine test boundaries (PASS Power Analysis and Sample Size Software, 2015). As the GA at delivery will be electronically extracted from medical records on all women, no adjustment has been made to account for loss to follow-up.

### Statistical Analysis

Statistical analysis will be performed on an intention-to-treat principle, with a secondary assessment of treatment received based on swabs collected at 26-28 weeks' GA. PTB outcomes between groups will be analysed using a z-test of proportions. Supplementary logistic regression analyses will be performed to examine group differences in sPTB rates and categorical secondary outcomes, while adjusting for the stratification factors and confounding due to maternal and/or pregnancy characteristics. Binary and nominal logistic regressions will be performed to evaluate the impact of the microbial risk factors used as screening criteria in the trial (PASS 2014). These regressions will also consider additional microbiological and immunological data, alone and together with maternal and pregnancy characteristics, to refine the risk factors for sPTB and derive risk equations for future implementation. All hypothesis tests will be two-sided with [symbol] =0.05.

### REFERENCES

(1) van't Hooft J, Duffy J M, Daly M, Williamson P R, Meher S, Thom E, Saade G R, Alfirevic Z, Mol B W, Khan K S; Global Obstetrics Network (GONet). (2016) A Core Outcome Set for Evaluation of Interventions to Prevent Preterm Birth. Obstet Gynecol. 127(1):49-58.
(2) Payne M S, Ireland D J, Watts R, Nathan E A, Furfaro L L, Kemp M W, Keelan J A, Newnham J P. (2016) Ureaplasma parvum genotype, combined vaginal colonisation with Candida albicans, and spontaneous preterm birth in an Australian cohort of pregnant women. BMC Pregnancy Childbirth. 16:312.
(3) Lamont R F. (2015) Advances in the prevention of infection-related preterm birth. Front Inflammation. 6:566.
(4) Kiss H, Petricevic L, Husslein P. (2004) Prospective randomised controlled trial of an infection screening programme to reduce the rate of preterm delivery. BMJ. 329:371.
(5) Kiss H, Petricevic L, Martina S, Husslein P. (2010) Reducing the rate of preterm birth through a simple antenatal screen-and-treat programme: A retrospective cohort study. Eur J Obstet Gynecol Reprod Biol. 153:38-42.
(6) Griffin C. (2015) Probiotics in obstetrics and gynaecology. Aust N Z J Obstet Gynaecol. 55:201-9.
(7) Parma M, Stella Vanni V, Bertini M, Candiani M. (2014) Probiotics in the prevention of recurrences of bacterial vaginosis. Alternative Therap Health Med. 20 Suppl. 1:52-7.
(8) Newnham J P, White S W, Meharry S, Lee H S, Pedretti M K, Arrese C A, Keelan J A, Kemp M W, Dickinson J E, Doherty D A. (2017) Reducing preterm birth by a statewide multifaceted program: an implementation study. Am J Obstet Gynecol. 216(5):434-442.
(9) Romero R, Dey S K, Fisher S J. (2014) Preterm labor: one syndrome, many causes. Science. 345(6198):760-5.
(10) Bahat Dinur A, Koren G, Matok I, Wiznitzer A, Uziel E, Gorodischer R, Levy A. Fetal safety of macrolides. Antimicrob Agents Chemother. 2013. 57(7):3307-11.
(11) Yi J, Yoon B H, Kim E C. (2005) Detection and biovar discrimination of Ureaplasma urealyticum by real-time PCR. Mol Cell Probes. 19(4):255-260.
(12) Ferandon C, Peuchant O, Janis C, Benard A, Renaudin H, Pereyre S, Bebear C. (2011) Development of a real-time PCR targeting the yidC gene for the detection of Mycoplasma hominis and comparison with quantitative culture. Clin Microbiol Infect. 17(2):155-9.
(13) Santiago G L, Deschaght P, El Aila N, Kiama T N, Verstraelen H, Jefferson K K, Temmerman M, Vaneechoutte M. (2011) Gardnerella vaginalis comprises three distinct genotypes of which only two produce sialidase. Am J Obstet Gynecol. 204(5):450.
(14) Cartwright C P, Lembke B D, Ramachandran K, Body B A, Nye M B, Rivers C A, Schwebke JR. (2012) Development and validation of a semiquantitative, multitarget PCR assay for diagnosis of bacterial vaginosis. J Clin Microbiol. 50(7):2321-9.
(15) Payne, M S, Tabone T, Kemp M W, Keelan J A, Spiller O B, Newnham J P. (2013) High-resolution melt PCR analysis for the genotyping of Ureaplasma parvum directly from clinical samples. J Clin Microbiol. 52(2):599-606.
(16) Klindworth A, Pruesse E, Schweer T, Peplies J, Quast C, Horn M, Glöckner F O. (2011) Evaluation of general 16S ribosomal RNA gene PCR primers for classical and next-generation sequencing-based diversity studies. Nucleic Acids Res. 41(1).
(17) Caporaso J G, Kuczynski J, Stombaugh J, Bittinger K, Bushman F D, Costello E K, Fierer N, Peña A G, Goodrich J K, Gordon J I, Huttley G A, Kelley S T, Knights D, Koenig J E, Ley R E, Lozupone C A, McDonald D, Muegge B D, Pirrung M, Reeder J, Sevinsky J R, Turnbaugh P J, Walters W A, Widmann J, Yatsunenko T, Zaneveld J, Knight R. (2010) QIIME allows analysis of high-throughput community sequencing data. Nat Methods. (5):335-6.
(18) Jensen J S, Bjornelius E, Dohn B, Lidbrink P. (2004) Use of TaqMan 5′ nuclease real-time PCR for quantitative detection of Mycoplasma genitalium DNA in males with and without urethritis who were attendees at a sexually transmitted disease clinic. J Clin Microbiol. 42(2):683-92.
(19) Innings A, Ullberg M, Johansson A, Rubin C J, Noreus N, Isaksson M, Herrmann B. (2007) Multiplex real-time PCR targeting the RNase P RNA gene for detection and identification of Candida species in blood. J Clin Microbiol. 45(3):874-80.

1. A method to determine if a pregnant woman is at risk of infection-associated spontaneous pre-term birth (sPTB), the method comprising the steps of:
a) testing a sample of vaginal fluid for the presence of the following bacteria:
i) Ureaplasma parvum genotype SV3 and/or Ureaplasma parvum genotype SV6;
ii) Gardnerella vaginalis; and
iii) Lactobacillus iners
wherein the presence of the bacteria indicates that the subject is at risk of sPTB.

2. The method of claim 1 wherein the testing method also tests for the presence of Fusobacterium nucleatum and wherein the presence of either:
Fusobacterium nucleatum in the absence of Ureaplasma parvum genotype SV3 and/or Ureaplasma parvum genotype SV6; or
Ureaplasma parvum genotype SV3 and/or Ureaplasma parvum genotype SV6, Gardnerella vaginalis, and Lactobacillus iners
indicates that the subject is at risk of a sPTB.

3. The method of claim 1 wherein the testing method is qPCR.

4. The method of claim 1 wherein the testing is carried out at between 10 and 24 weeks' gestation.

5. A method to determine if a pregnant woman would benefit from treatment to prevent infection-associated spontaneous pre-term birth (sPTB), the method comprising the steps of:
a) testing a sample of vaginal fluid for the presence of the following bacteria:
i) Ureaplasma parvum genotype SV3 and/or Ureaplasma parvum genotype SV6;
ii) Gardnerella vaginalis; and
iii) Lactobacillus iners
wherein the presence of the bacteria indicates that the subject is at risk of a sPTB and therefore would benefit from treatment to prevent sPTB.

6. A method to treat a pregnant woman at risk of infection-associated spontaneous pre-term birth (sPTB), comprising the steps of:
a) testing a sample of vaginal fluid for the presence of the following bacteria:
i) Ureaplasma parvum genotype SV3 and/or Ureaplasma parvum genotype SV6;
ii) Gardnerella vaginalis; and
iii) Lactobacillus iners
b) if the bacteria are present, giving the pregnant woman antibiotic therapy to eliminate the bacteria.

7. A method of reducing the risk of a pregnant woman having a infection-associated spontaneous pre-term birth (sPTB), comprising the steps of:
a) testing a sample of vaginal fluid for the presence of the following bacteria:
i) Ureaplasma parvum genotype SV3 and/or Ureaplasma parvum genotype SV6;
ii) Gardnerella vaginalis; and
iii) Lactobacillus iners
b) if the bacteria are present, giving the pregnant woman antibiotic therapy to eliminate the bacteria and therefore reduce the risk of sPTB.

8. A kit to determine if a pregnant woman is at risk of infection-associated spontaneous pre-term birth (sPTB), the kit comprising:
a) a means for testing a sample of vaginal fluid for the presence of the following bacteria:
i) Ureaplasma parvum genotype SV3 and/or Ureaplasma parvum genotype SV6;
ii) Gardnerella vaginalis; and
iii) Lactobacillus iners
b) instructions for use.

9. The method of any one of claims 5 to 7 or kit of claim 8 wherein the testing method also tests for the presence of Fusobacterium nucleatum and wherein the presence of either:
Fusobacterium nucleatum in the absence of Ureaplasma parvum genotype SV3 and/or Ureaplasma parvum genotype SV6; or
Ureaplasma parvum genotype SV3 and/or Ureaplasma parvum genotype SV6, Gardnerella vaginalis, and Lactobacillus iners
indicates that the subject is at risk of a sPTB.

10. The method of any one of claims 5 to 7 or kit of claim 8 wherein the testing method is qPCR.

11. The method of any one of claims 5 to 7 or kit of claim 8 wherein the testing is carried out at between 10 and 24 weeks' gestation.
